(19) United States
(12) Patent Application Publication
(10) Pub. No.: US 2023/0181660 A1
(43) Pub. Date: Jun. 15, 2023

(54) CARIBBEAN SEA MOSS TINCTURE

(71) Applicant: Zachary G. Rucker, JR., Nashville, TN (US)

(72) Inventor: Zachary G. Rucker, JR., Nashville, TN (US)

(21) Appl. No.: 18/066,035

(22) Filed: Dec. 14, 2022

Related U.S. Application Data
(60) Provisional application No. 63/265,386, filed on Dec. 14, 2021.

(51) Int. Cl. A61K 36/04; A61K 36/752; A61K 36/54; A61K 36/31; A61K 36/185; A61K 47/10

(52) U.S. Cl. CPC A61K 36/04; A61K 36/752; A61K 36/54; A61K 36/31; A61K 36/185; A61K 47/10

(57) ABSTRACT

A tincture composition includes gel-free sea moss extract; about 80-90% by volume glycerin; about 10-18% by volume ethanol; and about 0.5-1.5% cinnamon oil. The total amount of glycerin, ethanol, and cinnamon oil is 100%. A method of producing the composition includes soaking sea moss in water; drying the sea moss; immersing the sea moss fully in glycerin; providing an extraction blend; blending the sea moss, the glycerin, and the extraction blend to form a suspension; straining the suspension to extract a tincture; and heating the tincture at 50-200 degrees Fahrenheit for 1-24 hours. The extraction blend includes, per 1 part by volume glycerin: 1/16 to 2 parts by volume ethanol and 1/100 to ¼ parts by volume of cinnamon oil. The tincture composition has an excellent shelf life and improved palatability. A binding agent may be added to prevent separation.

### CROSS-REFERENCE TO RELATED APPLICATION

This application claims the benefit of priority of U.S. provisional application No. 63/265,386, filed Dec. 14, 2021, the contents of which are herein incorporated by reference.

### BACKGROUND OF THE INVENTION

The present invention relates to tincture and, more particularly, to a Caribbean Sea Moss tincture.

Many health benefits are attributed to the consumption of sea moss including but not limited to thyroid health, immunity, gut health, weight loss, heart health, blood sugar management, and fertility. For example, it may be used to remove pain and inflammation throughout the body and to treat cancer, e.g., tumors. However, sea moss is not easily consumed. It is typically sold raw, dried, or in a powdered form. Consumers are then left to find creative ways of consuming the sea moss and adding it to their diet, such as through smoothies.

Much attention is given to Irish sea moss, but Irish sea moss is just one of many sea mosses available. Caribbean sea moss is often overlooked.

As can be seen, there is a need for an easy, convenient way to consume sea moss products and receive their health benefits.

### SUMMARY OF THE INVENTION

In one aspect of the present invention, a tincture composition comprises gel-free sea moss extract; about 80-90% by volume glycerin; about 10-18% by volume ethanol; and about 0.5-1.5% cinnamon oil, where the total amount of glycerin, ethanol, and cinnamon oil is 100%.

In another aspect of the present invention, a method of producing a nutritional supplement comprises soaking sea moss in water; drying the sea moss; immersing the sea moss fully in glycerin; providing an extraction blend comprising, per 1 part by volume glycerin: 1/16 to 2 parts by volume ethanol and 1/100 to ¼ parts by volume of cinnamon oil; blending the sea moss, the glycerin, and the extraction blend to form a suspension; straining the suspension to extract a tincture; and heating the tincture at 50-200 degrees Fahrenheit for 1-24 hours.

Advantageously, the tincture comprises a mixture of alcohol and sea moss for positive effects on a user's general health and well-being. The composition is beneficial as a topical healing agent for and inflammation remover from wounds including cuts and bruises; skin problems, acne, eczema, age wrinkles, skin moisturizer; hair growth, and hair shine and as a mouthwash and breath freshener for both humans and animals. Due to the novel extraction method, the beneficial agents of the sea moss are more readily bioavailable than in prior art raw, dried, powdered, water-extracted, and glycerin-extracted products and thus has a faster and stronger effect.

These and other features, aspects and advantages of the present invention will become better understood with reference to the following description and claims.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

A general overview of the various features of the invention will be provided, with a detailed description following. Broadly, an embodiment of the present invention provides a composition for a health supplement in a form of a tincture. The composition may comprise a sea moss tincture formed by carrageenan extraction with ethanol.

In some embodiments, the composition may comprise about 80-90% by volume glycerin, such as about 85%.

In some embodiments, the composition may comprise about 10%-18% by volume ethanol, such as about 14%.

In some embodiments, the composition may comprise about 0.5%-1.5% by volume cinnamon oil or other liquid cinnamon extract, such as about 0.8%.

The tincture may be taken orally. The nutritional supplement may be dropped or placed into a user's mouth, such as placed under the user's tongue to enter the bloodstream quickly. An example of a dose is 1 full dropper administered 1-3 times per day. Alternatively, the tincture may be applied topically. The benefits of the tincture may include boosting energy levels, supplementing vitamins, natural weight loss, building muscle, and boosting an immune system.

The present invention may be produced with any suitable sea moss. In some embodiments, Caribbean sea moss is preferred, in particular varieties of sea moss harvested in the region of St. Lucia, an island in the Caribbean. These varieties are believed to have superior nutritional properties to Irish sea moss. The sea moss may be a Caribbean sea moss selected from the group consisting of multi color, purple, green, gold dried herb, and any sea moss on a full spectrum of St. Lucia Sea moss. Full spectrum sea moss comprises a blend of sea mosses of various colors.

Rinsed and dried sea moss may be immersed in glycerin prior to further processing. In order to prevent gel formation, the sea moss is fully immersed. In some embodiments, the glycerin is preferably vegetable glycerin.

The nutritional supplement may be made by a method according to an embodiment of the present invention, comprising: soaking sea moss in water; drying the sea moss; immersing the sea moss in glycerin, the amount of which may be determined to be 1 part by volume of the liquid ingredients; an extraction blend comprising 1/16 to 2 parts by volume alcohol to 1 part of glycerin, the alcohol being any ethanol-containing potable liquid such as grain alcohol, vodka, and a combination thereof; and ¼ to 8 parts by volume of botanical oil selected from the group consisting of coconut oil, cinnamon oil, virgin olive oil, rosemary oil, oregano oil, lavender oil, chamomile oil, castor oil, and combinations thereof; blending the sea moss and the extraction blend to form a suspension; straining the suspension to extract a tincture; and evaporating the tincture at 50-200 degrees Fahrenheit for 1-24 hours. For example, a Rotovap® laboratory chemical reactor or an oil dispenser may be used to heat the tincture composition. In some embodiments, a citrus juice such as lime juice, or in some cases lecithin, in an amount of 1/32 to 1/16 parts by volume may be added as a binding agent before evaporating the tincture to prevent separation. The resulting blend may then be poured into jars.

Some embodiments of the composition may be made with the following steps: A sea moss may be soaked in an alkaline water solution or distilled water. Lime or lime juice may be added to the water or water solution. The sea moss may be soaked for 24-48 hours in a container to remove any salt and/or fishy taste. The type of container is not particularly limited; a plastic or glass container may suffice. The sea moss may be removed from the water or water solution. The sea moss may then be rinsed to remove any dirty particles or debris. Alkaline water, distilled water, or a water solution may be used to rinse the sea moss. Additionally, dirt, particles, and debris may be removed by hand. The sea moss may be dried, for example for 24-48 hours. The sea moss may be dried in a dry room and/or in a container with a lid slightly cracked, enabling some air to circulate into the container. When the sea moss has dried, approximately 4 oz (ounces) of sea moss may be immersed in glycerin, after which at least one alcohol and, in some cases, botanical oils, may be added. The ingredients may be mixed in a blender for about one to five minutes, such as about three to five minutes, forming a blended mix.

Alcohol surprisingly extracts carrageenan in a liquid form, unlike products extracted using water or glycerin, which form a gel. The liquid product is superior to the gel for a variety of reasons. The liquid product is better preserved and has a much longer shelf life than the prior art gel product, which must be refrigerated and lasts about 2 weeks. Stored in a cool, dry place, the shelf life may be a year or two, depending upon the alcohol content. The liquid product is much faster to produce, as the prior art gel product takes about 25 to 26 hours to produce. It is more convenient and has a more palatable taste and texture. Moreover, it serves as a better topical composition, e.g., for wounds.

In some embodiments, dried raw Moringa leaf may be added to the sea moss prior to blending. For example, about 0.5 parts by weight Moringa leaf may be combined with about 1 part by weight sea moss. Without being bound to theory, Moringa is believed to render nutrients within the moss more available or more effective. The resulting tincture may sometimes be referred to herein as MoringaMoss.

In some embodiments, hemp leaf or bud may be combined with the sea moss and Moringa prior to blending. For example, about 0.5 parts by weight of hemp leaf may be combined with about 1 part by weight sea moss. In alternative embodiments, CBD oil may be added to the sea moss and Moringa, the blended mix, or the resulting tincture. The resulting tincture may sometimes be referred to herein as MoringaMossCBD.

The blended mix may be strained. For example, the mix may be poured from the blender over a large micron strainer bag until the mix no longer exits the strainer. The blended mix may then be poured back into a blender, blended again, then strained again.

The blended mix may be inserted into a rotary evaporator or into an oil diffuser. 1-2 tablespoons of lime juice may be added. The blended mix may be evaporated or diffused at a temperature of approximately 50 degrees Fahrenheit (10° Celsius) to about 200 degrees Fahrenheit (93.3° Celsius) and for at least about 1 hour up to about 24 hours to produce a tincture. For example, the composition may be cooked at 30 degrees Celsius to 60 degrees Celsius from 30 minutes to 4½ hours. The tincture may be placed into any size tincture bottle or container suitable for storing the tincture.

In some embodiments, additional ingredients may also be added to the blend. Such includes may include but are not limited to: moringa leaf, hemp, vegetable glycerin, sugarcane ethanol, ethanol, grain alcohol, vodka alcohol, moonshine alcohol, flavored oils such as those obtained from LorAnn® Oils, Inc. of Lansing, Mich., cinnamon leaf oil, extra virgin olive oil, virgin olive oil, rosemary oil, oregano oil, lavender oil, chamomile oil, castor oil, basil oil, coconut oil, flaxseed oil, hemp oil, lemon juice, lemon oil, lime juice, lime oil, mushroom oil, cinnamon bark oil, cannabis oil, cannabis plant, hemp plant, senna plant, senna oil, cassia plant, cassia oil, black cumin plant, black cumin oil, Plantago rugelii plant, Plantago rugelii oil, basil plant, sunflower plant, sunflower oil, dandelion plant, dandelion oil, black seed plant, black seed oil, Cannabis ruderalis plant, Cannabis ruderalis oil, bamboo leaf, nettle leaves, betel leaves, and any combination thereof. For example, various ingredients may be added for flavor, such as cinnamon leaf extract oil.

Cinnamon leaf extract, cinnamon oil, and cinnamon bark oil are surprisingly effective to neutralize the ocean taste of sea moss. Cinnamon extract and/or oil also serves to improve carrageenan extraction and to preserve the resulting tincture. The combination of alcohol with cinnamon extract provides surprisingly superior preservation of the product.

The liquid sea moss product may be combined with an additional ingredient selected from the group consisting of blackseed oil or Nigella sativa extract (black cumin seed), Kratom plant, clove plant, pumpkin seeds, hemp plant, basil leaf, turmeric, ginger, Pumpkin seeds, kratom plant, garlic plant, clove plant, black cumin seeds, turmeric, ginger, honey, sage, and any combination thereof.

### EXAMPLES

Examples 1-4 in the following table illustrate compositions according to embodiments of the present invention. The sea moss was purple Caribbean Sea Moss in each example.

| Ingredient | Example 1 | | Example 2 | | Example 3 | | Example 4 | |
|---|---|---|---|---|---|---|---|---|
| Dried sea moss | 4 | oz | 4 | oz | 6 | oz | 6 | oz |
| Sugarcane | — | | ½ | cup | ½ | cup | ½ | cup |
| ethanol | | | | | | | | |
| Grain alcohol | 1/16-2 | cups | + | | + | | + | |
| Vodka | 1/16-2 | cups | + | | + | | + | |
| Ethanol | — | | + | | + | | + | |
| Moonshine | — | | + | | + | | + | |
| alcohol | | | | | | | | |
| Vegetable | 2 | cups | 2 | cups | 4 | cups | 5 | cups |
| glycerin | | | | | | | | |

| | | | | | | | |
|---|---|---|---|---|---|---|---|
| Cinnamon leaf | — | ⅛ | cup | ¼ | cup | ¼-⅓ | cup |
| extract | | | | | | | |
| Concentrated | — | 1-2 | Tbsp | 1-2 | Tbsp | ½ | cup |
| lemon juice | | | | | | | |

| gluten-free | — | 1-2 full | 1-2 full | | 5 full | |
|---|---|---|---|---|---|---|
| flavored oil | | droppers | droppers | | droppers | |
| Moringa leaf | — | — | 2 | oz | 2 | oz |

| | | | | | |
|---|---|---|---|---|---|
| Hemp | — | — | — | 2 | oz |

| | | | | | |
|---|---|---|---|---|---|
| Coconut oil | 1/16-2 | cups | — | — | — |
| Cinnamon oil | 1/16-2 | cups | — | — | — |
| Virgin olive oil, | 1/16-2 | cups | — | — | — |
| cold-pressed | | | | | |
| Rosemary oil | 1/16-2 | cups | — | — | — |
| Oregano oil | 1/16-2 | cups | — | — | — |
| Lavender oil | 1/16-2 | cups | — | — | — |
| Chamomile oil | 1/16-2 | cups | — | — | — |
| Castor oil | 1/16-2 | cups | — | — | — |

+ indicates the presence of an alcohol without indicating an amount.

It should be understood, of course, that the foregoing relates to exemplary embodiments of the invention and that modifications may be made without departing from the spirit and scope of the invention as set forth in the following claims.

What is claimed is:

1. A tincture composition comprising gel-free sea moss extract; about 80-90% by volume glycerin; about 10-18% by volume ethanol; and about 0.5-1.5% cinnamon oil, where the total amount of glycerin, ethanol, and cinnamon oil is 100%.

2. The tincture composition of claim 1, wherein the gel-free sea moss extract is derived by immersing rinsed and dried sea moss in glycerin before adding ethanol and cinnamon oil.

3. The tincture composition of claim 1, further comprising lime juice.

4. The tincture composition of claim 1, further comprising botanical oils selected from the group consisting of coconut oil, olive oil, rosemary oil, oregano oil, lavender oil, chamomile oil, castor oil, black seed oil, and combinations thereof.

5. The tincture composition of claim 1, further comprising Moringa extract.

6. The tincture composition of claim 5, further comprising hemp extract.

7. The tincture composition of claim 1, further comprising an ingredient selected from the group of kratom plant, bamboo leaf, and combinations thereof.

8. A method of producing a nutritional supplement comprising:
soaking sea moss in water;
drying the sea moss;
immersing the sea moss fully in glycerin;
providing an extraction blend comprising, per 1 part by volume glycerin: 1/16 to 2 parts by volume ethanol and 1/100 to ¼ parts by volume of cinnamon oil;
blending the sea moss, the glycerin, and the extraction blend to form a suspension;
straining the suspension to extract a tincture; and
heating the tincture at 50-200 degrees Fahrenheit for 1-24 hours.

9. The method of claim 8, wherein the extraction blend further comprises botanical oils selected from the group consisting of: coconut oil, cinnamon oil, virgin olive oil, rosemary oil, oregano oil, lavender oil, chamomile oil, castor oil, and any combination thereof.

10. The method of claim 8, further comprising adding 1/30 to 1/16 parts by volume of a binding agent before evaporating the tincture.
